(19) United States
(12) Patent Application Publication
(10) Pub. No.: US 2006/0121597 A1
(43) Pub. Date: Jun. 8, 2006

(54) Cytoblock preparation system and methods of use

(76) Inventor: Rongshan Li, Brookfield, WI (US)

Correspondence Address: RYAN KROMHOLZ & MANION, S.C., POST OFFICE BOX 26618, MILWAUKEE, WI, 53226, US

(21) Appl. No.: 11/284,501

(22) Filed: Nov. 22, 2005

Related U.S. Application Data
(60) Provisional application No. 60/630,870, filed on Nov. 24, 2004.

(51) Int. Cl. C12M 1/00

(52) U.S. Cl. 435/283.1

(57) ABSTRACT

A system for preparing a cytoblock includes an instrument and a series of associated supplies. The instrument provides all of the mechanical instrumentation necessary for preparation of the cytoblock in a single piece of laboratory equipment. In one embodiment, the instrument includes a centrifuge, a supernatant and moisture removal device, an incubation chamber, and a mixing device. The associated supplies may be provided in the form of a kit and include a centrifuge tube containing a fixative, a matrix container containing a matrix material, a transfer tube, a tamping device, a tissue cassette, an embedding tray, and an embedding block. A method of preparing a cytoblock using the kit. The cytoblock can include a single cell population, or can include several distinct cell populations.

### RELATED APPLICATION

This application claims the benefit of U.S. Provisional Patent Application Ser. No. 60/630,870, filed 24 Nov. 2004, and entitled “Cytoblock Preparation System and Methods of Use.”

### FIELD OF THE INVENTION

The invention relates to a system and method for the separation and preparation of cells and/or tissue for microscopic examination. In particular, the invention relates to cell block preparation for immunocytochemistry studies of fine needle aspirates.

### BACKGROUND OF THE INVENTION

Fine needle aspiration (FNA) is a widely used screening diagnostic procedure. However, only a small and finite amount of material can be obtained by FNA. The current process in the clinical laboratory does not maximally use this limited amount of material. As a result, there is typically only enough material obtained to perform initial or screening tests. The limited amount of material collected through this procedure largely inhibits further classification of the disease, which results in more invasive procedures for a more conclusive diagnosis. This not only results in increased costs, but significantly delays the diagnosis as well.

The need remains for systems and methods which maximize the use of this limited material for different immunocytochemistry (ICC) studies to permit a more conclusive diagnosis to be made by a single FNA procedure alone and without the need for more invasive procedures.

### SUMMARY OF THE INVENTION

An object of the invention is to provide a cytoblock preparation system that provides the necessary instrumentation to process a FNA for microscopic examination in a single piece of laboratory equipment. The system permits the preparation of multiple sections from a single FNA. Each section contains sufficient cells or material for staining or other studies. In one embodiment, the system provides a centrifuge, a vacuum device, a temperature incubation chamber, and a mixing device.

Another object of the invention is to provide a kit for use with the system which supplies the components required for processing of the specimen. In one embodiment, the kit provides a centrifuge tube containing a fixative, a matrix container containing a matrix material, a transfer tube with a tamping device, a tissue cassette, an embedding tray with a divider, and paraffin or other embedding material.

### BRIEF DESCRIPTION OF THE DRAWINGS

FIG. 1 is a schematic block diagram of an instrument for the preparation of a cytoblock.

FIG. 2 is a schematic of a kit of components for use with the instrument shown in FIG. 1.

FIG. 3 is a schematic of a centrifuge tube containing a fixative solution to which cellular material has been added.

FIG. 4 is a schematic block diagram illustrating the centrifugation of the centrifuge tube shown in FIG. 3.

FIG. 5 is a schematic block diagram illustrating removal of supernatant from the centrifuged specimen by use of a moisture removal device.

FIG. 6 is a schematic illustrating transfer of the cellular pellet from the centrifuge tube to a transfer tube after removal of the supernatant.

FIG. 7 is a schematic block diagram illustrating the transfer of the cellular pellet from the transfer tube into a matrix container containing a pre-warmed matrix material using a tamping device.

FIG. 8 is a schematic block diagram illustrating the mixing of the matrix material and the cellular pellet using a mixing probe.

FIG. 9 is a schematic block diagram illustrating the cooling incubation of the matrix material/cell pellet mixture to form a gelled specimen.

FIG. 10 is a schematic illustrating the transfer of the gelled specimen from the matrix container to the transfer tube.

FIG. 11A is a schematic illustrating the transfer of the gelled specimen from the transfer tube to a chamber within a tissue cassette using the tamping device.

FIG. 11B is a schematic illustrating an alternative embodiment of a tissue cassette in which the chamber is removable.

FIG. 12 is a schematic illustrating the placement of the gelled specimen within a well within an embedding block.

FIGS. 13A-13C are perspective views illustrating various configurations of embedding blocks and wells.

FIG. 14 is a schematic illustrating the placement of an embedding tray over the embedding block containing the gelled specimen.

FIG. 15 is a schematic illustrating the placement of the embedding tray on a warming plate.

FIG. 16 is a schematic illustrating the placement of the embedding tray on a cooling plate

FIG. 17 is a schematic of an alternative embodiment of a matrix container in which the matrix container takes the form of a syringe.

FIG. 18 is a schematic view of an embedding tray including a dividing insert.

FIGS. 19A-19C are schematic views of additional embodiments of the transfer tube of the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures.

FIG. 1 shows a device or instrument 10 for the preparation of a cytoblock. The instrument 10 provides the mechanical equipment necessary to process a fine needle aspiration (FNA) specimen for microscopic examination in a single piece of laboratory equipment. In the illustrated embodiment, the instrument 10 provides a centrifuge 12, supernatant and moisture removal device 14, temperature incubation chamber 16, mixing device 18, a warming plate 60, and a cooling plate 19. In the preferred embodiment the instrument 10 is integrally connected and unitary in its overall nature. However, it is also contemplated that any combination of two or more of the abovementioned components could be formed together as a unitary structure as is convenient in the laboratory setting.

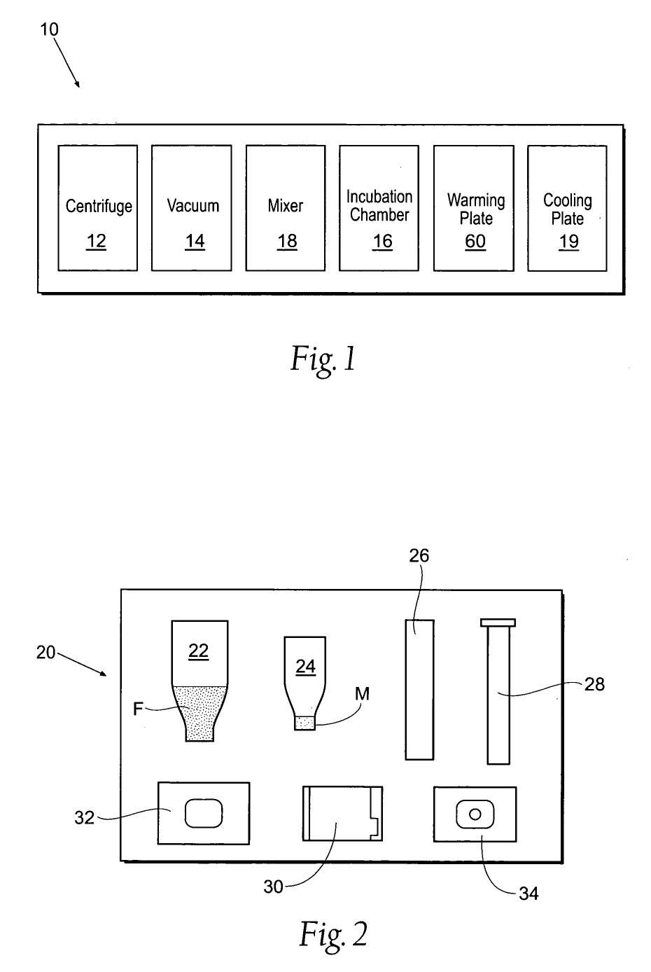

The instrument 10 is designed for use with additional materials, including disposable and/or consumable components, required for processing of the specimen, which are desirably supplied in the form of a kit 20, as FIG. 2 shows. In the illustrated embodiment, the kit 20 provides a centrifuge tube 22 containing a fixative F, a matrix container 24 containing a matrix material M, a transfer tube 26, a tamping device 28, a tissue cassette 30, an embedding tray 32, and a paraffin or other embedding block 34. It is contemplated that a kit 20 may be provided that includes any or all of these items and that the items may be provided in any desired quantity. Although in the illustrated kit 20, the transfer tube 26 and the tamping device 28 are shown separately, it is also contemplated that these two items could be provided together with the tamping device 28 inserted in the transfer tube 26, ready for use in preparing a cytoblock sample.

The instrument 10 and associated kit 20 are particularly well-suited for use in preparing and processing specimens for immunocytochemistry (ICC) studies on the limited materials collected by FNA, biopsy, endoscopic procedures, washings, and lavages and therefore will be described in accordance with such use. It will be readily apparent, however, that the instrument 10 and kit 20 are also suitable for use in other studies, e.g., special stains, in situ hybridization, RNA and DNA studies, as well as in basic research requiring the collection and saving of treated cells.

Together, the instrument 10 and kit 20 provide a system that permits the preparation of multiple sections from a single FNA. Each section contains sufficient cells or material for staining or other studies.

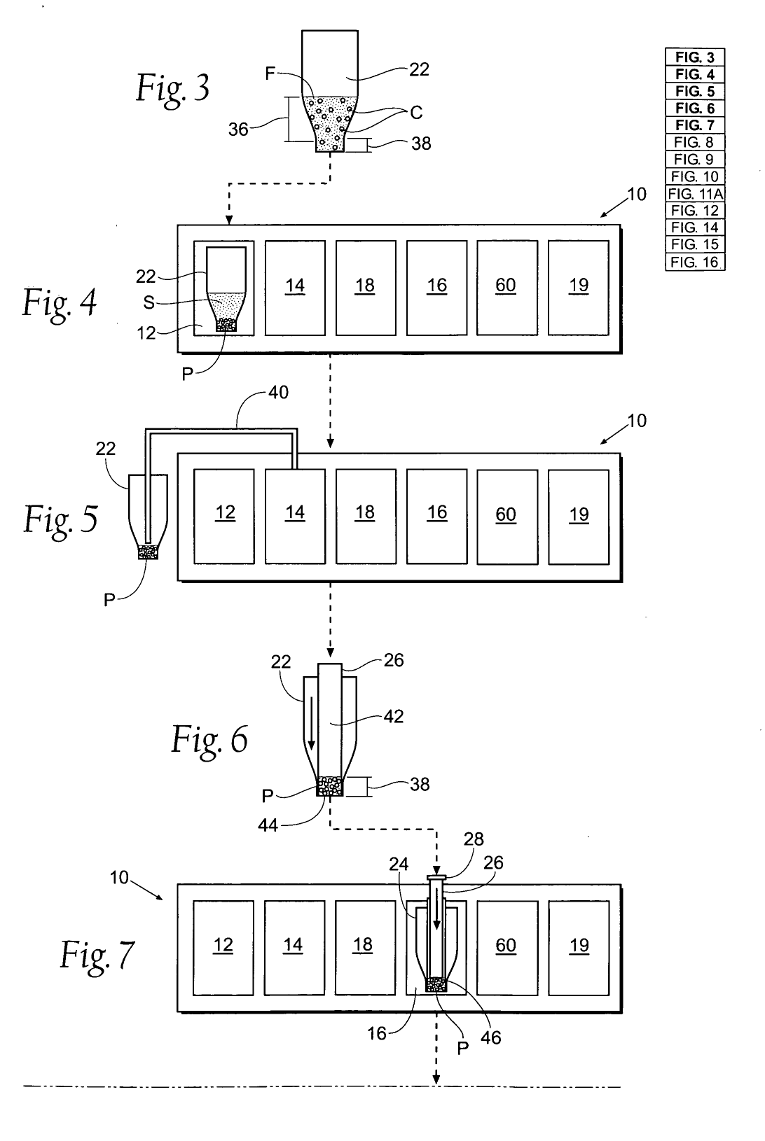

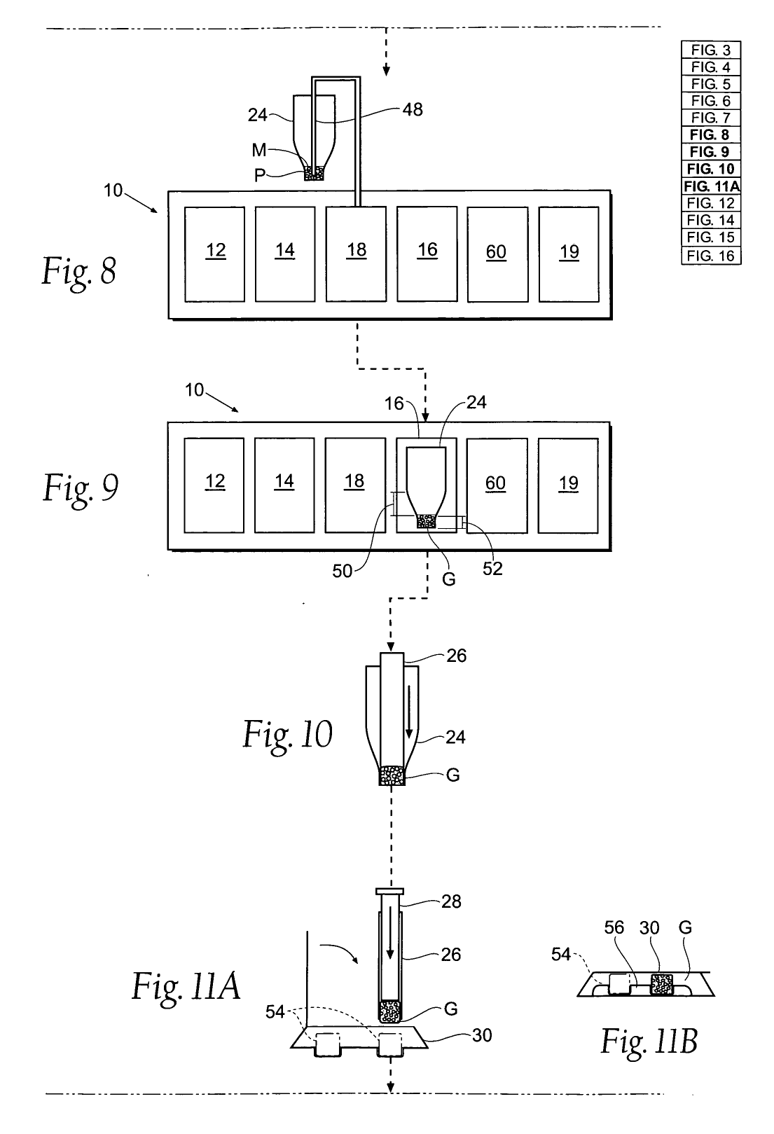

In use, as shown in FIG. 3, the cellular material C obtained from the FNA or other specimen is added to the centrifuge tube 22 containing the fixative F, e.g., formalin. Desirably, the tube 22 has a tapered region 36 and a non-tapered, reduced diameter bottom region or chamber 38. This configuration allows for maximal concentration of cells or specimen at the bottom of the tube 22 after centrifugation. As will be explained later, together with transfer tube 26 and tamp 28, this configuration permits easy removal and transfer of essentially all of the cellular material.

It should be noted that it is also contemplated that the cellular material C could be added to a centrifuge tube 22 which does not already contain a predetermined quantity of fixative. In such an embodiment, an appropriate quantity of fixative would be added to the centrifuge tube 22 along with the cellular material C.

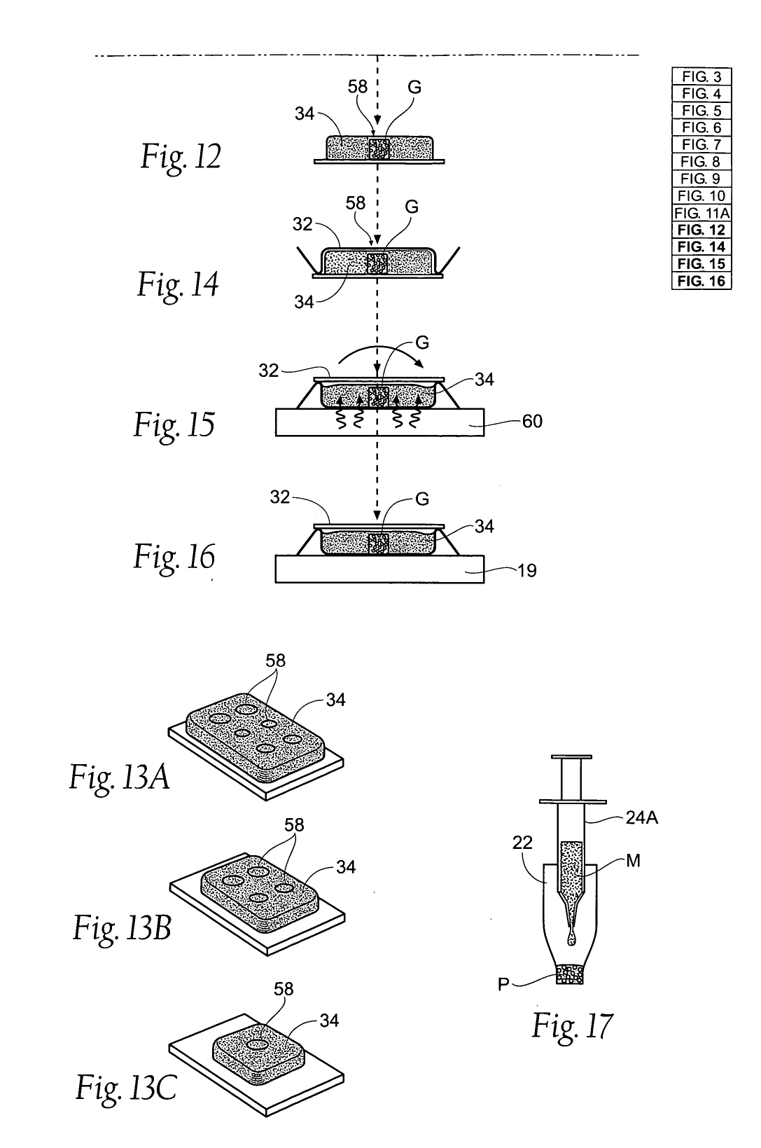

The tube 22 is then placed into the centrifuge 12 for separation. In a representative embodiment, the centrifuge 12 is a conventional low speed centrifuge 12 that permits the separation of the aspirate/fixative mixture into a supernatant S and a cell pellet P, as seen in FIG. 4.

The supernatant S is then removed, as shown in FIG. 5, using supernatant and moisture removal device 14, e.g., with aspiration tubing 40 or other aspiration means, such that tube 22 retains the pellet P. It is important to remove as much of the supernatant S as possible without disturbing the pellet P. In the preferred embodiment the supernatant and moisture removal device 14 comprises a vacuum device, however alternate methods of removing the supernatant S are contemplated, including, but not limited to, using laser or heat provided by the instrument 10. The instrument 10 can also include a detector to sense the amount of moisture in the tube 22.

The matrix container 24 (which contains a viscous matrix mixture M is desirably pre-heated by placing the container 24 in the temperature incubation chamber 16. A variety of matrixes are available in the art, which include agar, agarose gel or “histogel” solid at ambient temperature, Methocell®, Matrix Gel®, OCT compounds, paraffin, denatured and non-denatured collagen, fibronectin, laminin, and mixtures thereof. Those skilled in the art will know of other suitable matrixes for cell immobilization, or will be able to ascertain such, without undue experimentation. The incubation chamber 16 may be a single chamber selectively adjustable over a broad range of temperature, e.g., between −50-100° C. Alternatively, the chamber 16 may include distinct heating and cooling chambers (e.g., a separate heating block and cold plate) that are independently adjustable within a defined temperature range, e.g., 50-100° C. and 2-8° C. respectively.

The matrix material M is pre-heated by selecting a temperature that permits liquefaction of the matrix. The incubation chamber 16 includes a well (not shown) or otherwise receives the matrix container 24 to heat the matrix material M to the desired temperature prior to adding the matrix material M to the cell pellet P. It will be readily apparent that the chamber may include a series of wells, which may be of the same or of different size and/or configuration, to accommodate multiple specimens and/or containers 24 of varying size or shape. In one embodiment, the temperature of the chamber 16 is first set to between 90-100° C. to liquefy the matrix material M. Once the matrix material M has been liquefied, the temperature is adjusted and lowered to 50° C. to maintain the matrix material M in the liquid state.

The pellet P is then removed from the centrifuge tube 22 by use of transfer tube 26 and tamp 28. The transfer tube 26 is a tube having a hollow core 42 and open end 44. The transfer tube 26 is placed into the centrifuge tube 22 and passed over the pellet P to retain the pellet within the hollow core 42, as shown in FIG. 6. The transfer tube 26 preferably has a complementary size and shape to the chamber 38 and thereby permits the collection and transfer of essentially the entire pellet P. The tamping instrument 28 desirably has end portion 46 and is sized and configured for passage through the transfer tube 26 to release the pellet P into the matrix container 24, as shown in FIG. 7. Although the tamp 28 could be formed as a solid piece, the preferred embodiment of the tamp 28 includes a hollow channel along the length of the tamp 28, passing through the center of the tamp 28. The hollow channel extends through the end portion 46. This hollow channel allows air to be released through the tamp 28. In this arrangement, the matrix container 24 desirably contains a pre-measured amount of matrix material to provide a desired ratio of matrix material M to the cellular pellet P, e.g., a 1:1 ratio.

The tube 26 and tamp 28 may be reusable, e.g., formed of metal, or may be suitable for disposal after a single use, e.g., formed of plastic. Additional embodiments of the transfer tube 26 and tamp 28 are shown in FIGS. 19a-19c. FIG. 19a shows a transfer tube 126 and tamp 128 which is similar to the preferred embodiment, however, the tamp 128 has a narrow central portion. The tamp and transfer tube operate in the same manner as the preferred embodiment, wherein the tamp is pushed to advance the tamp inside the transfer tube and the tamp is pulled to retract the tamp from the tube.

FIG. 19b is similar to the embodiment of FIG. 19a, however the transfer tube 226 and tamp 228 include a mating screw mechanism such that the tamp 228 is advanced in the transfer tube 226 by rotating the tamp 228 in one direction and retracted from the transfer tube 226 by rotating the tamp 228 in the opposite direction.

FIG. 19c is similar to the preferred embodiment, however a spring mechanism is engaged between the transfer tube 326 and the tamp 328. In this embodiment, the tamp 328 is advanced by pushing on the tamp 328 to engage the spring. The tamp 328 is retracted from the tube 326 by again pushing on the tamp 328 to disengage the spring. This mechanism is similar to that utilized in a spring-retractable ball point pen. While the preferred embodiment discloses utilizing the transfer tube 26,126,226,326 and tamp 28,128,228,328 to transfer the cell specimen, it is also contemplated that the tube 26,126,226,326 and tamp 28,128,228,328 could have additional uses in the medical field, such as for taking dermatological biopsies.

The pellet P is then thoroughly resuspended and mixed within the matrix material M using the mixing device 18. In the illustrated embodiment, the mixing device 18 provides a mixing probe 48, which can be positioned within and near the bottom of the tube 24 and activated to provide mechanical stirring or mixing motion. It will be readily apparent that a variety of other mixing means may be provided, e.g., a vortex.

With reference now to FIG. 9, the tube 24 is then placed in the incubation chamber 16 for a time period sufficient to solidify the specimen into a gel G. The chamber 16 receives the tube 24 to cool the matrix material M/cell pellet P mixture to the desired temperature. The temperature is desirably selectively adjustable within a range that permits solidification or gelling of the histogel, preferably from −2 to −8° C., and more preferably about −4° C.

The matrix tube 24 desirably provides a tapered region 50 and reduced diameter chamber 52 similar to centrifuge tube 22. Chamber 52 serves to form and maintain the gelled specimen G in a desired shape or configuration. In a preferred embodiment, the chamber 52 is of a round or cylindrical configuration and results in the formation of an essentially round or circular gelled specimen G. It is to be understood that the chamber 52 may be variously configured to provide a gelled specimen G of a desired size and shape, e.g., square or oval.

After solidification, as shown in FIG. 10, the gelled specimen G is removed from the tube 24 using the transfer tube 26 and tamp 28 or other removal means. The transfer tube 26 is placed into the tube 24 and passed through the specimen G. The tube 26 retains the specimen G within the hollow core 42, much like a straw that has been passed through solid gelatin. The transfer tube 26 preferably has a complementary size and shape to the chamber 52 and thereby permits the collection and transfer of essentially the entire gelled specimen G and serves to retain the specimen G in the desired configuration. The tamping instrument 28 can then be passed through the core 42 to release the gelled specimen G, which can then be further processed, e.g., by frozen section or by embedding in a paraffin or other embedding block. It should be noted that while paraffin is the preferred embedding material, and is used throughout the description of this process, one of skill in the art will recognize that any suitable embedding material may be utilized. Commonly employed embedding materials include, but are not limited to nitrocellulose, glue, denatured or non denatured collagen, fibronectin, laminin, gum syrup, OTC compounds, and various formulations of plastic polymers.

In processing the specimen G by embedding, the specimen G is then placed into the tissue cassette 30. The cassette 30 may be formed of plastic or other any other suitable material, and may be adapted for multiple or single use. As shown in FIG. 11A, the cassette 30 desirably includes a recessed well or chamber 54 to receive the specimen G. Additional chambers 54 may be provided for additional specimens or quality control samples as desired. In an alternative embodiment, illustrated in FIG. 11B, a removable basket 56 providing one or more chambers 54 is provided. The chamber 54 extend through the removable basket 56, forming aperture in the basket 56. A lid or other cover (not shown) may be provided to cover the chamber 54 and further secure the specimen G within the cassette 30. The chamber 54 is preferably similar and complementary in size and configuration to the chamber 38 and specimen G so as to retain the specimen G in the desired configuration during subsequent processing.

The removable basket 56 preferably has at least one cylindrical chamber 54 integrally formed therein. However, it will also be readily apparent that the size, number, and configuration of the chambers 54 of the basket 56 may be varied to accommodate the procedures being performed and the number and types of specimens being processed. The removable basket 56 could be made of any suitable material, including, but not limited to plastic or foam.

After processing, the specimen G is transferred from the cassette 30 into a pre-bored hole or well 58 within the embedding block 34, as FIG. 12 shows. Block 34 is desirably provided with at least one pre-bored hole to receive the prepared gelled specimen G. FIGS. 13A-13C provide, by way of example and not limitation, possible configurations of embedding blocks 34 and wells 58. It will be readily apparent that the block 34 may be of any suitable size and configuration, e.g., rectangular (FIGS. 13A and 13B) or square (FIG. 13C). It will also be readily apparent that the size, number, and configuration of wells 58 may be varied to accommodate the procedures being performed and the number and types of specimens being processed, e.g., a single block 34 may provide wells 58 of different sizes (FIG. 13A).

In an alternative embodiment, block 34 may be provided in kit 20 without pre-bored wells 58. In this arrangement, transfer tube 26 is preferably formed of metal or otherwise adapted to bore through the block 34 to form a well 58 or series of wells 58 so that the number and placement of wells 58 may be determined by user.

The embedding tray 32, which is desirably complementary in size and shape to block 34, is placed over the block (FIG. 14). In the illustrated embodiment, the tray 32 is a conventional embedding tray and may be formed of metal, plastic or other suitable material and may be suitable for single or multiple use.

The tray 32, containing block 34 with specimen G, is then inverted and placed on the warming plate 60 (FIG. 15) or otherwise warmed to provide sufficient liquefaction to fill and essentially eliminate well 58 and thereby embed the specimen G. The temperature of the warming plate 60 is desirably selectively adjustable within a range that permits sufficient liquefaction, e.g., from 55 to 65° C.

In an alternative embodiment, wells 58 may be closed and the specimen G firmly embedded by pipetting or otherwise delivering heated, liquefied paraffin without use of the tray 32 (not shown). The paraffin may be pre-heated and liquefied by placing on warming plate 60, microwaving, or other suitable means.

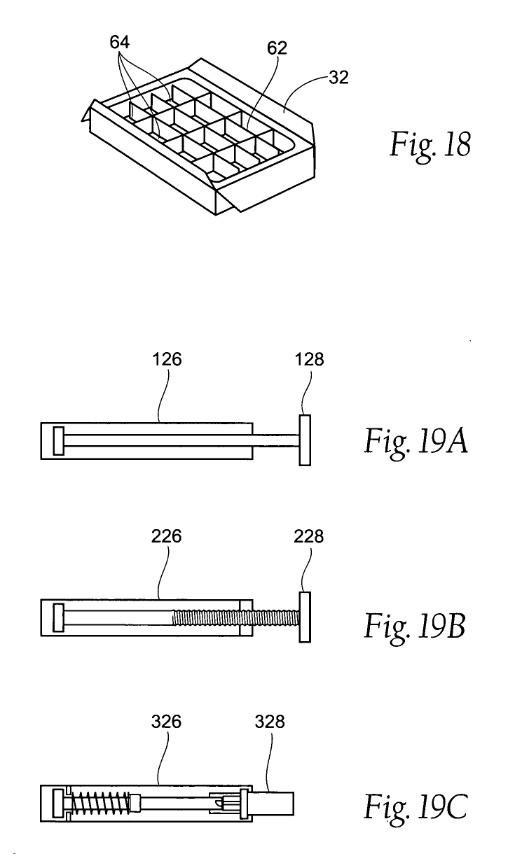

In an additional alternative embodiment, the tray 32 may be provided with a partitioned insert 62 which includes multiple divisions 64, as shown in FIG. 18. In use, the insert 62 is first placed in the embedding tray 32. At least one specimen G may then be placed in each partition 64 of the insert 62. The specimen G is embedded by pipetting or otherwise delivering heated liquefied paraffin to the tray 32. The tray 32 is then cooled to form a solidified block 34. Alternatively, the tray 32 can be filled with paraffin before the insert 62 is placed in the tray 32. After the insert 62 is placed in the paraffin on the tray 32, at least one specimen G can be placed in each partition 64 of the insert. The tray 32 is then cooled to form a solidified block 34.

The tray insert 62 may have any suitable number and configuration of divisions 64. The tray insert 62 may be made of any suitable material, including, but not limited to plastic or metal. This configuration would be particularly useful in creating a cell array containing cell samples from multiple origins, as is further described below.

The block 34 can then be placed on the cooling plate 19 or otherwise cooled to solidify the block 34 (FIG. 16). The temperature of the cooling plate 19 is desirably selectively adjustable within a range that permits solidification, e.g., from −50 to +4° C. The block 34 may then be and removed from tray 32 for further cytological or histological processing, e.g., cutting of the prepared block 34 and preparation of slides for staining or other diagnostic techniques. The cooperating and complementary components of the described system, in particular the chambers 38 and 52, transfer tube 26 and tamp 28, and well 58 serve to retain the embedded specimen G in the desired shape, e.g., cylindrical. Because the desired shape has been maintained throughout processing of the specimen, a consistent and uniform number of cells or material is provided on each slide. As a result, more diagnostic procedures may be performed from a single FNA or other specimen, reducing the need to obtain additional specimens and thereby also reducing the need for more invasive procedures. However, it can be appreciated that although a cylindrical shape is preferred, the configuration can be in any suitable shape, provided that the chambers 38 and 52, transfer tube 26 and tamp 28 and well 58 are all formed with the same desired shape.

FIG. 17 illustrates an alternative embodiment of a matrix container 24A in which the container 24A takes the form of a syringe. Matrix container 24A may be placed in the heating device 16 or otherwise pre-warmed to liquefy the matrix material M. The desired amount of matrix material M may then be delivered directly into the centrifuge tube 22 containing the pellet P (see also FIG. 5). In this arrangement, the container 24A may include sufficient material M for preparing more than one sample and designed for reheating and reuse. In one embodiment, the incubation chamber 16 includes a well (not shown) or other means for holding the container 24A during both warming and delivery of the material M. The pellet P is then thoroughly mixed and cooled within the centrifuge tube 22 as previously described with reference to FIGS. 8 and 9 respectively. The gelled specimen G may then be transferred to the cassette 30 using the transfer tube 26 and tamp 28, as also previously described with reference to FIG. 10. It is further contemplated that the matrix material could have a dye added, so that the paraffin is readily distinguishable from the cell mixture.

While the preferred embodiment of the invention utilizes cells obtained by fine needle aspiration, it should be clear to one of skill in the art that cellular material captured by other means could also be utilized to create a cytoblock. Cell material could also be collected by endoscopy, including but not limited to arthroscopy, bronchoscopy, colonoscopy, colposcopy, cystoscopy, ERCP (endoscopic retrograde cholangiopancreatograthy), EGD (esophogealgastroduodensoscopy), endoscopic biopsy, gastroscopy, laparoscopy, laryngoscopy, proctoscopy and thoracoscopy. Cells could also be obtained from lavage procedures, including but not limited to bronchoalveolar, breast ductal, nasal, pleural, peritoneal, gastrointestinal, arthroscopic, and urinary bladder lavages. It is also contemplated that cells could be collected from catheters such as those used in infusion, cardiovascular, rental, bladder, urothral, hemodynamic monitoring, neurological, and other procedures which would be obvious to one of skill in the art.

It is difficult to screen the expression level of a gene or molecule in different cell lines, especially for newly described ones. The current routine methods for this purpose include western blot, immunocytochemical study using fluorescence-labeled antibodies, real-time RT-PCR, northern blot, in-situ hybridization, etc.

The current sources of cells for research include commercial or privately-maintained sources of viable cells in culture, frozen viable cells of specific cell lines, and primary cultured cells derived from different organs/tissues from different organisms, plants, animals and/or human. It is very difficult and expensive to maintain these cells for scientists and researchers. A “Fixed or Permanent Cell Bank” may be provided to improve the current system and forms of cell sources. In this system, all cells from different possible sources (commercial companies, primary cultured cells derived from animals or other sources, etc.) are cultured, collected, fixed with a fixative (formalin, alcohol, et. al) and embedded in paraffin or other materials to form a long-lasting (permanent) form of cell source. Based on this principle, different cells can embedded individually like an individual account, and different cell cultures can be together to form a “Cell Bank”.

It is contemplated that a variety of cell lines can be collected and embedded in paraffin blocks 34. Cultured cells may first be embedded in paraffin by conventional or by the above-described methods.

A portion of the paraffin embedded cells may then be taken out by various methods (e.g., by use of transfer tube 26 and tamp 28) and re-embedded in a paraffin block 34 as above-described to generate paraffin-embedded cell blocks 34 in a fashion of tissue microarray. It is preferred that the method of embedding including the embedding tray and partitioned tray insert is utilized to create the cell array.

Various types of arrays could be created by the above described method. By way of example, and not limitation, these types of arrays include embryonic cell array, adult cell array, primary cell array, cell line array, tissue array, mammalian array, zoo array, personal cell array, genetically altered array, chemically treated array, or disease cell array. Further it is contemplated to create a cell array by the above described method wherein the different cell mixtures differ in one or more of the characteristics selected from the group consisting of genotypic characteristics, species, origin, developmental stage, developmental origin, tissue origin, chemical treatment, cell-cycle point and disease state.

The blocks 34 may contain different combinations of different cells from different systems and organs. By way of example and not limitation, different breast cancer cell lines can be provided in one block, different carcinoma cell lines in one block, different sarcoma cell lines in one block, different benign cell lines in one block, different epithelial cell lines in one block, and different mesenchymal cell lines in one block. It is also contemplated to create a cell array with cell populations from several different types of body tissues in one cell array, the tissues including but not limited to blood, muscle, nerve, brain, heart, lung, liver, pancreas, spleen, thymus, esophagus, stomach, intestine, kidney, testes, ovary, hair, skin, bone, breast, uterus, bladder, spinal cord, and body fluids.

Cells from many cell lines, including cells from primary cultures, cells from humans, rats, mice, and other animals, cells from different organisms, and cells from an organism at different stages of development, may thereby be provided in a single cell block. The cells may be treated with different conditions (different chemicals, different temperatures, different culture conditions, etc.) based on specific requirements, collected, and embedded in a single block 34.

A variety of cell lines may be maintained as a “cell bank” and blocks 34 containing specific cell lines may be pre-formed and provided as “ready to use” blocks 34 to researchers or others. Pre-made blocks 34 including the desired embedded specimens or cell lines may be customized (e.g., specific cell line(s) and number of wells) and manufactured according to the user's specific needs.

Sections can then be generated from different blocks 34 and slides containing the cells from these sections can be obtained and processed as desired, e.g., protein, DNA, RNA, or other studies.

The foregoing is considered as illustrative only of the principles of the invention. Furthermore, since numerous modifications and changes will readily occur to those skilled in the art, it is not desired to limit the invention to the exact construction and operation shown and described.

I claim:

1. An apparatus for use in preparing a cytoblock comprising
a centrifuge;
a supernatant and moisture removal device;
a temperature incubation chamber;
a mixing device;
a warming plate;
a cooling plate;
wherein said centrifuge, said supernatant and moisture removal device, said temperature incubation chamber, said mixing device, said warming plate, and said cooling plate are integrally connected together in a single piece of laboratory equipment.

2. An apparatus as in claim 1 wherein said supernatant and moisture removal device further comprises a vacuum device with aspiration means connected thereto.

3. An apparatus as in claim 1 wherein said supernatant and moisture removal device is a laser.

4. An apparatus as in claim 1 wherein said supernatant and moisture removal device is a heat source.

5. A kit for preparing a cytoblock said kit comprising a combination of at least one centrifuge tube;
at least one matrix container;
at least one transfer tube;
at least one tamping device; and
at least one tissue cassette.

6. The kit of claim 5 further including
at least one embedding tray and
at least one embedding block.

7. The kit of claim 5 wherein said centrifuge tube contains a fixative solution.

8. The kit of claim 5 wherein said matrix container contains a matrix material.

9. The kit of claim 7 wherein said matrix container is in the form of the syringe.

10. The kit of claim 5 wherein said tissue cassette includes at least one integrally formed chamber.

11. The kit of claim 5 where in said tissue cassette includes a removable insert, said removable insert having at least one integrally formed chamber.

12. The kit of claim 6 wherein said embedding tray includes a removable divider.

13. The kit of claim 6 wherein said embedding tray includes an integrally formed divider.

14. A method of preparing a cytoblock comprising
preparing a cell pellet;
combining said cell pellet with a matrix material to form a cell mixture;
processing said cell mixture in a tissue cassette;
embedding the processed cell mixture in an embedding block.

15. The method of claim 14 wherein the preparing step further comprises
depositing cell material in a centrifuge tube;
adding a predetermined amount of fixative to the centrifuge tube;
placing said centrifuge tube in a centrifuge device to create a cell pellet and supernatant; and
removing said supernatant from said centrifuge tube.

16. The method of claim 14 wherein the preparing step further comprises
depositing cell material in a centrifuge tube, said centrifuge tube containing an appropriate amount of fixative;
placing said centrifuge tube in a centrifuge device to create a cell pellet and supernatant; and
removing said supernatant from said centrifuge tube.

17. The method of claim 16 where in the cell material is obtained by fine needle aspiration.

18. The method of claim 16 where the cell material is obtained by endoscopy.

19. The method of claim 16 where the cell material is obtained by lavage.

20. The method of claim 16 where the cell material is obtained by a catheter.

21. The method of claim 14 wherein said combining step further comprises
providing a matrix container containing a matrix material;
heating said matrix container to liquefy said matrix material;
transferring said cell pellet from said centrifuge tube to said matrix container;
mixing said cell pellet and said matrix material to create a suspension; and
cooling said matrix container to solidify the matrix material and create a cell mixture.

22. The method of claim 14 wherein said combining step further comprises
providing a matrix container containing a matrix material, said matrix container having the form of a syringe;
heating said matrix container to liquefy said matrix material;
depositing a predetermined amount of liquefied matrix material into said centrifuge tub;
mixing said cell pellet and said matrix material to create a suspension; and
cooling said centrifuge tube to solidify the matrix material and create a cell mixture.

23. The method of claim 14 wherein said processing step further comprises transferring said cell mixture from said matrix container to a tissue cassette and processing said tissue cassette.

24. The method of claim 14 wherein said embedding step further comprises
transferring at least a portion of said cell mixture from said tissue cassette to an embedding block, said embedding block being made of a wax and formed with at least one hole therein to receive at least one cell mixture;
placing an embedding tray over the embedding block and inverting the embedding tray and embedding block;
warming said embedding tray and embedding block to at least partially melt said embedding block and embed said cell mixture in said embedding block; and
cooling said embedding tray and embedding block to solidify said embedding block.

25. The method of claim 14 wherein said embedding step further comprises
transferring at least a portion of said cell mixture from said tissue cassette to an embedding tray, said embedding tray being provided with a partitioned insert formed with at least one partition therein to receive at least one cell mixture;
depositing a predetermined amount of embedding material into said embedding tray to embed said cell mixture into an embedded block, said embedding material being heated to liquefy said embedding material; and
cooling said embedding tray solidify said embedding block.

26. The method of claim 14 wherein said embedding step further comprises
transferring at least a portion of said cell mixture from said tissue cassette to an embedding tray, said embedding tray is provided with liquefied embedding material therein and a portioned insert formed with at least one partition therein to receive at least one cell mixture; and
cooling said embedding tray to create a solidified embedded block.

27. A method of preparing a cytoblock comprising
depositing cell material in a centrifuge tube, said centrifuge tube containing an appropriate amount of fixative;
placing said centrifuge tube in a centrifuge device to create a cell pellet and supernatant;
removing said supernatant from said centrifuge tube;
providing a matrix container containing a matrix material;
heating said matrix container to liquefy said matrix material;
transferring said cell pellet from said centrifuge tube into said matrix container;
mixing said cell pellet and said matrix material to create a suspension;
cooling said matrix container to solidify the matrix material and create a cell mixture;
transferring said cell mixture from said matrix container into a tissue cassette;
processing said tissue cassette;
transferring at least a portion of said cell mixture from said tissue cassette to an embedding tray, said embedding tray being provided with a partitioned insert formed with at least one partition therein to receive at least one cell mixture;
depositing a predetermined amount of embedding material into said embedding tray to embed said cell mixture into an embedded block, said embedding material being heated to liquefy said embedding material; and
cooling said embedding tray solidify said embedding block.

28. A method of preparing a cytoblock comprising
depositing cell material in a centrifuge tube, said centrifuge tube containing an appropriate amount of fixative;
placing said centrifuge tube in a centrifuge device to create a cell pellet and supernatant;
removing said supernatant from said centrifuge tube;
providing a matrix container containing a matrix material, said matrix container having the form of a syringe;
heating said matrix container to liquefy said matrix material;
depositing a predetermined amount of liquefied matrix material into said centrifuge tube;
mixing said cell pellet and said matrix material to create a suspension;
cooling said centrifuge tube to solidify the matrix material and create a cell mixture;
transferring said cell specimen from said centrifuge tube to a tissue cassette;
processing said tissue cassette;
transferring at least a portion of said cell mixture from said tissue cassette to an embedding tray, said embedding tray being provided with a partitioned insert formed with at least one partition therein to receive at least one cell mixture;
depositing a predetermined amount of embedding material into said embedding tray to embed said cell mixture into an embedded block, said embedding material being heated to liquefy said embedding material; and
cooling said embedding tray solidify said embedding block.

29. A method of creating a cell array comprising
providing an embedding tray with a divider insert, an embedding material, and a plurality of embedded cell mixtures;
transferring each of said cell mixtures to a separate portion of said embedding tray;
embedding said cell mixtures in said embedding tray to create a composite block with a plurality of distinct cell mixtures therein.

30. The method of claim 29 wherein the embedding step further comprises delivering heated liquefied embedding material to the embedding tray.

31. The method of claim 29 wherein each cell mixture contains a population of viable cells of a specific type that are contained and immobilized within the cell mixture.

32. The method of claim 31 wherein each cell mixture contains cells of a unique type.

33. The method of claim 31 wherein at least one cell mixture contains a cell population that is unique with respect to at all other cell mixture cell populations

34. The method of claim 31 wherein the cell array is an embryonic cell array, adult cell array, primary cell array, cell line array, tissue array, mammalian array, zoo array, personal cell array, genetically altered array, chemically treated array, or disease cell array.

35. The method of claim 31 wherein the cell array is a cancer cell array.

36. The method of claim 31 wherein the cells contained in the different cell mixtures differ in one or more of the characteristics selected from the group consisting of genotypic characteristics, species, origin, developmental stage, developmental origin, tissue origin, chemical treatment, cell-cycle point and disease state.

37. The method of claim 36 wherein the cells contained in the different cell mixtures differ in species of origin.

38. The method of claim 36 wherein cells contained in the different cell mixtures differ in developmental origin, said developmental origin being selected from the group consisting of endodermal, mesodermal, and ectodermal origin.

39. The method of claim 36 wherein cells contained in the different cell species differ in tissue origin.

40. The method of claim 37 wherein species of origin is selected from the group consisting or human, mouse, rat, fruit fly, worm, yeast and bacterium.

41. The method of claim 39 wherein said tissue origin is selected from the group consisting of blood, muscle, nerve, brain, heart, lung, liver, pancreas, spleen, thymus, esophagus, stomach, intestine, kidney, testis, ovary, hair, skin, bone, breast, uterus, bladder, spinal cord, and body fluids.
